(19) United States
(12) Patent Application Publication
(10) Pub. No.: US 2025/0382651 A1
(43) Pub. Date: Dec. 18, 2025

(54) PROCESS FOR RECOVERING AND PURIFYING VANILLIN

(71) Applicants: DANSTAR FERMENT AG, Zug (CH); INTERNATIONAL FLAVORS & FRAGRANCES INC., New York, NY (US)

(72) Inventors: Oliver FRANKOVIC, Reinach (CH); Mariangela MORTATO, Reinach (CH); Gerhard Michael LOBMAIER, Reinach (CH)

(21) Appl. No.: 18/838,517

(22) PCT Filed: Feb. 14, 2023
(86) PCT No.: PCT/US2023/062578

§ 371 Date: Aug. 14, 2024

Related U.S. Application Data
(60) Provisional application No. 63/310,395, filed on Feb. 15, 2022.

(51) Int. Cl. C12P 19/46; B01J 20/20; B01J 39/05; B01J 41/07; B01J 47/014; C12N 9/42; C12P 19/14

(52) U.S. Cl. CPC C12P 19/46; B01J 20/20; B01J 39/05; B01J 41/07; B01J 47/014; C12N 9/2445; C12P 19/14; C12Y 302/01021

(57) ABSTRACT

This disclosure relates to processes for recovering and purifying vanillin from a microbial fermentation broth, wherein the fermentation broth comprises a vanillin conjugate, such as vanillin glucoside, which is produced during the microbial fermentation by a microbial cell that is capable of producing and secreting the vanillin conjugate.

### CROSS-REFERENCE TO RELATED APPLICATIONS

This application claims priority to and the benefit of U.S. Provisional Application Ser. No. 63/310,395, filed on Feb. 15, 2022, the contents of which are incorporated herein in their entirety, to the extent permitted by law.

### FIELD

This disclosure relates to processes for recovering and purifying vanillin from a microbial fermentation broth, wherein the fermentation broth comprises a vanillin conjugate, such as vanillin glucoside, which is produced during the microbial fermentation by a microbial cell that is capable of producing and secreting the vanillin conjugate.

### DESCRIPTION OF THE INVENTION

1) In a first embodiment the disclosure relates to a process for recovering and purifying vanillin from a microbial fermentation broth, wherein the fermentation broth comprises a vanillin conjugate which is produced during the microbial fermentation by a microbial cell that is capable of producing and secreting the vanillin conjugate, said process comprising the steps of:
(I)(a) separating and removing the microbial cells from the fermentation broth such that a liquid remains that is substantially free of microbial cells followed by converting the vanillin conjugate that is contained in the liquid into vanillin and the corresponding conjugation partner, or
(I)(b) converting the vanillin conjugate that is contained in the fermentation broth into vanillin and the corresponding conjugation partner followed by separating and removing the microbial cells from the fermentation broth such that a liquid remains that is substantially free of microbial cells;
followed by either:
(II)(a) adding a polar organic solvent miscible with water to the liquid resulting from step (I)(a) or (I)(b), wherein the liquid resulting from step (I)(a) is preferably filtered either before or after addition of the polar organic solvent;
(II)(b) treating the vanillin solution produced in step (II)(a) with either:
   (i) a cation exchange adsorbent followed by a weak base anion exchange adsorbent, or
   (ii) a weak base anion exchange adsorbent followed by a cation exchange adsorbent; and
(II)(c) optionally decolorizing the resulting solution with an adsorbent;
   or:
(II′)(a) treating the liquid resulting from step (I)(a) or (I)(b) with a non-ionic adsorbent, wherein the liquid resulting from step (I)(a) is preferably first filtered before treatment with the non-ionic adsorbent, wherein the non-ionic adsorbent used in step (II′)(a) is capable of adsorbing vanillin and wherein the treatment comprises the steps of (i) adsorption of the vanillin under conditions that allow vanillin to bind to the non-ionic adsorbent and (ii) desorption of the bound vanillin into a solution;
(II′)(b) treating the obtained vanillin solution with either:
   (i) a cation exchange adsorbent,
   (ii) a weak base anion exchange adsorbent, or
   (iii) both a cation exchange adsorbent and a weak base anion exchange adsorbent in either order; and
(II′)(c) optionally decolorizing the resulting solution with an adsorbent; and
(III) crystallizing the vanillin from the obtained solution.

As used herein, by “microbial cell” is meant a prokaryotic or eukaryotic cell, preferably selected from bacteria, fungi, and especially yeast.

The term “vanillin” refers to the compound with the chemical name 4-hydroxy-3-methoxybenzaldehyde.

The term “vanillin conjugate” refers to vanillin covalently bonded to a further molecular entity (herein called “conjugation partner”) wherein the conjugation partner can be any molecular entity which is suitable for production and secretion by a microbial cell in the form of a vanillin conjugate containing such molecular entity and wherein the conjugation partner can be separated from the vanillin by conversion of the vanillin conjugate into vanillin and the corresponding conjugation partner. Conjugation partners include but are not limited to a sugar, such as a poly-, di-, or mono-saccharide, preferably a monosaccharide, such as very preferably D-glucose. In a very preferred embodiment the term “vanillin conjugate” therefore refers to “vanillin glucoside”.

The term “vanillin glucoside” refers to the compound “vanillin 4-O-β-D-glucoside” which also called “vanillin β-D-glucoside”.

A microbial cell useful according to the present invention can be a prokaryotic or eukaryotic cell that is capable of producing and secreting a vanillin conjugate, such as especially vanillin glucoside, into the fermentation medium such as especially a cell that has been genetically modified to be capable of producing and secreting a vanillin conjugate, such as especially vanillin glucoside. Such genetically modified microbial cells are for example described in WO 2004/111254, Hansen et al., Appl. Environ. Microbiol. 75 (9): 2765-2774 (2009), WO 2013/022881, WO 2015/009558, and WO 2021/022216.

The separation and removal of the microbial cells from the fermentation broth in step (I)(a) or (I)(b) such that a liquid remains that is substantially free of microbial cells can be carried out by any suitable separation techniques. Generally, this will be achieved by centrifugation followed by filtration or by membrane separation processing. A suitable membrane separation technique is either microfiltration, ultrafiltration, nanofiltration, or a combination thereof, whereas suitable techniques for the filtration after centrifugation include pressure filtration and vacuum filtration. Where pressure filtration is used, this may be carried out using any suitable apparatus such as a candle filter or a filter press. Preferably, filtration is performed by the addition of a filter aid. A filter aid can be either added to the suspension to be filtered (e.g., 0.1-5% w/w) or placed on the filter as a precoat through which the liquid must pass. Any agent consisting of solid particles that improves filtering efficiency can be used. Preferably, filtration materials are based on cellulose, perlite, or diatomite.

In case of membrane separation processing, preferably ultrafiltration is used. Biomass separation can be performed by ultrafiltration using membranes with a nominal molecular weight cut-off (MWCO) in the range of 1-100 kDa, preferably 1-10 kDa. The flux rates and/or yields used through these various membranes may be similar. However, smaller molecular weight cut-offs may be preferred as they eliminate more impurities. On the other hand, finer ultrafilters will require cleaning more frequently requiring process shutdown so there is a balance to be made in the selection of the optimum molecular weight cut-off to achieve an acceptable purity without compromising the process economics by frequent shutdowns for cleaning. Preferably, composite fluoropolymer membranes are used, such as ETNA01PP (1 kDa MWCO) or ETNA10PP (10 kDa MWCO) from Alfa Laval. Diafiltration can be performed with ≥1-2, preferably 1.6, volume of demineralized water versus volume of concentrated broth (retentate).

The conversion of the vanillin conjugate into vanillin and the corresponding conjugation partner in step (I)(a) or (I)(b) can be carried out either by chemical conversion (e.g., hydrolysis) or enzymatic conversion, preferably by enzymatic conversion using a suitable enzyme under suitable conditions. Such enzymes are known in the art, such as for example β-glucosidases which can catalyze the conversion of vanillin glucoside into vanillin and glucose. In a preferred embodiment, the conversion of vanillin glucoside into vanillin and glucose is carried out using 0.002-0.1 g of a β-glucosidase, having an activity of about 10 enzyme units (u)/g, per 1 g of vanillin glucoside (corresponds to about 20-1'000 u/kg of vanillin glucoside), preferably 0.03-0.07 g/g (corresponds to about 300-700 u/kg of vanillin glucoside) at a pH of about 3.0-6.5, preferably 4.5-5.5, and at a temperature of about 30-60° C., preferably 50-55° C., preferably for 20-48 h.

Optionally, before conversion of the vanillin conjugate into vanillin and the corresponding conjugation partner in step (I)(a), the liquid is concentrated. The liquid can be concentrated with a volumetric concentration factor (VCF) ≥2. Preferably, the concentration is performed by reverse osmosis or wiped thin-film evaporator.

The concentration can be carried out by Reverse Osmosis at a temperature of about 10-50° C. or evaporation at about 50-80° C., preferably at about 60-65° C. Thin film composite membranes made of thin film composite polymer on polypropylene with >98% rejection measured on 2000 ppm NaCl at 25° C. and 16 barg can be used (e.g., Alfa Laval RO98pHt). Alternatively, Alfa Laval RO99 membranes, made of thin film composite polymer on polyester with ≥98% rejection measured on 2000 ppm NaCl at 25° C. and 9 barg can be used (e.g., Alfa Laval RO99).

A suitable polar organic solvent to be used in step (II)(a) is an organic solvent, or a mixture of different organic solvents, that is miscible in water, increases the solubility of vanillin, and is compatible to be used with the ion exchange adsorbents used in step (II)(b). In a preferred embodiment the polar organic solvent is an alcohol, or a mixture of different alcohols, preferably containing 1-6 carbon atoms in linear or branched form, such as e.g., a primary alcohol, unsaturated or especially saturated, such as e.g., ethanol and propanol such as 2-propanol. In an especially preferred embodiment, the polar organic solvent is ethanol or 2-propanol, most preferably ethanol. The amount of the polar organic solvent added to the liquid from step (I)(a) or (I)(b) is preferably such that it results in at least about 10%, 20%, 30%, 40%, 50%, 60%, or 70% v/v of the polar organic solvent compared to the total volume of the resulting solution. Very preferably the polar organic solvent constitutes about 20-50% v/v, especially about 20-40% v/v, most preferably about 20% v/v of the resulting solution.

Unless used regarding temperatures, the term “about” placed before a numerical value “X” preferably refers in the current application to an interval extending from X minus 10% of X to X plus 10% of X. In the particular case of temperatures, the term “about” placed before a temperature “Y” preferably refers in the current application to an interval extending from the temperature Y minus 5° C. to Y plus 5° C.

The filtration according to step (II)(a) or (II′)(a) can be carried out by any suitable filtration technique such as pressure filtration or vacuum filtration. Preferably, filtration is performed by the addition of a filter aid. A filter aid can be either added to the suspension to be filtered (e.g., 0.1-1% w/w) or placed on the filter as a precoat through which the liquid must pass. Any agent consisting of solid particles that improves filtering efficiency can be used. Preferably, filtration materials are based on cellulose, perlite, or diatomite.

Any suitable cation exchange adsorbent can be used in step (II)(b) or (II′)(b). It can be a week acid or strong acid cation exchange adsorbent. Preferably, the adsorbent used is a resin. The cation exchange adsorbent is preferably a strong acid cation exchange adsorbent, such as especially a strong acid cation exchange resin, preferably in H-Form. Preferably, macroporous resins are used. For example, macroporous polystyrenic resins and polystyrenic gel resins can be used, such as for example macroporous polystyrene resins that are crosslinked with divinylbenzene and have sulfonic acid as functional group.

Any suitable weak base anion exchange adsorbent can be used in step (II)(b) or (II′)(b). Preferably, the weak base anion exchange adsorbent is a weak base anion exchange resin, such as especially a weak base anion exchange macroporous resin. Especially, a weak base anion exchange macroporous resin with either a polystyrene or polyacrylic ester frame and a primary-tertiary amino group as the functional group can be used. While macroporous polystyrenic resins are preferred, polystyrenic gel resins can also be used. For example, gel-type resins with polyacryl crosslinked with divinylbenzene with a tertiary amine as functional group are suitable. In a very preferred embodiment the weak base anion exchange adsorbent, such as especially the weak base anion exchange resin, is converted to the Ac-form (acetate form) before being used. To do so, it can be treated with a solution of acetic acid of 5%, washed with demineralized water and preconditioned with the solvent of the feed.

Any suitable non-ionic (neutral) adsorbent can be used in step (II′)(a). Preferably, the non-ionic adsorbent, is a non-ionic resin, especially a macroporous non-ionic resin, and especially a hydrophobic resin. Polystyrenic, polyphenolic, or polymethacrylic non-ionic resins can be used. For example, macroporous polydivinylbenzene resins without functional groups are suitable.

Adsorption of the vanillin under conditions that allow vanillin to bind to the non-ionic adsorbent in step (II′)(a)(i) can be carried out by pumping the solution through a column containing the non-ionic adsorbent, preferably at flow rates ≤4 BV/h.

Desorption of the bound vanillin into a solution in step (II′)(a)(ii) can be carried out by pumping for example 2-4 BV, preferably 3 BV, of eluent through a column containing the non-ionic adsorbent, preferably at flow rates ≤4 BV/h. A suitable eluent for the desorption is an organic solvent, a mixture of different organic solvents, a mixture of water and an organic solvent, or a mixture of water and different organic solvents, provided such eluent increases the solubility of vanillin compared to water alone and is compatible to be used with the non-ionic adsorbent used in step (II′)(a). In a preferred embodiment the organic solvent is an alcohol, or a mixture of different alcohols, preferably containing 1-6 carbon atoms in linear or branched form, such as e.g., a primary alcohol, unsaturated or especially saturated, such as e.g., ethanol and propanol such as 2-propanol. In an especially preferred embodiment, the organic solvent is ethanol or 2-propanol, most preferably ethanol. The amount of the organic solvent is preferably at least about 40%, 50%, 60%, or 70% v/v compared to the total volume of the eluent. Very preferably the organic solvent constitutes about 50-90% v/v, especially about 70-80% v/v, most preferably about 80% v/v of the total volume of the eluent. In a very preferred embodiment, the eluent is aqueous ethanol with a concentration of 50-90% v/v, especially about 70-80% v/v, most preferably about 80% v/v ethanol.

The adsorbent used in optional step (II)(c) or (II′)(c) for decolorizing the solution can be carbon, especially activated carbon, preferably granular activated carbon. The solution can for example be pumped through a carbon column at flow rates ≤4 BV/h and at a temperature of about 15-25° C. The carbon requirement is preferably about 0.05-2 kg, more preferably about 0.3-1 kg, and most preferably about 0.5 kg, of carbon per kg of vanillin.

Before performing the crystallization in step (III), the vanillin solution can be polished by filtration to remove any traces of adsorbents. Such filtration can be performed using suitable filters, preferably with a pore size of ≤1 μm.

The crystallization in step (III) can be performed by concentration of the vanillin solution, for example up to a total dry matter of about 10-60%, preferably about 25-35%, by evaporation, e.g., under reduced pressure, such as at 0.1-0.4 barg, and at a temperature of about 50-55° C. The crystallization of vanillin is preferably performed at a pH of about 3.5-5.5, preferably about 4-5.5.

At the end of the evaporation, the vanillin solution can be cooled, for example to about 25-35° C., to initiate crystallization. If crystallization does not occur, seeding with pure vanillin can be performed. Seeding might be avoided if after concentration by evaporation no more than about 7% v/v of the organic solvent is present in the vanillin solution, which usually leads to spontaneous crystallization following cooling of the concentrated solution to for example about 25-35° C.

The present disclosure provides the following further embodiments:
2) In one embodiment the disclosure relates to the process of embodiment 1), wherein the vanillin conjugate is vanillin glucoside.
3) In another embodiment the disclosure relates to the process of embodiment 1) or 2), wherein the microbial cell is a fungal cell.
4) In another embodiment the disclosure relates to the process of embodiment 3), wherein the fungal cell is a yeast cell.
5) In another embodiment the disclosure relates to the process of embodiment 4), wherein the yeast cell is selected from Saccharomyces cerevisiae.
6) In another embodiment the disclosure relates to the process of any one of embodiments 1) to 5), wherein separation and removal of the microbial cells from the fermentation broth in step (I)(a) or (I)(b) such that a liquid remains that is substantially free of microbial cells is carried out by centrifugation followed by filtration or by membrane separation processing.
7) In another embodiment the disclosure relates to the process of embodiment 6), wherein separation and removal of the microbial cells is carried out by centrifugation followed by filtration.
8) In another embodiment the disclosure relates to the process of embodiment 6), wherein separation and removal of the microbial cells is carried out by ultrafiltration.
9) In another embodiment the disclosure relates to the process of any one of embodiments 1) to 8), wherein the conversion of the vanillin conjugate into vanillin and the corresponding conjugation partner in step (I)(a) or (I)(b) is carried out either by chemical conversion (e.g., hydrolysis) or enzymatic conversion.
10) In another embodiment the disclosure relates to the process of embodiment 9), wherein the conversion of the vanillin conjugate into vanillin and the corresponding conjugation partner is carried out by enzymatic conversion.
11) In another embodiment the disclosure relates to the process of embodiment 10), wherein the vanillin conjugate is vanillin glucoside and the enzymatic conversion is carried out by using a β-glucosidase as enzyme.
12) In another embodiment the disclosure relates to the process of any one of embodiments 1) to 11), wherein the process comprises step (I)(a).
13) In another embodiment the disclosure relates to the process of any one of embodiments 1) to 12), wherein before conversion of the vanillin glucoside conjugate into vanillin and the corresponding conjugation partner in step (I)(a), the liquid is concentrated, preferably with a volumetric concentration factor ≥2.
14) In another embodiment the disclosure relates to the process of any one of embodiments 1) to 13), wherein the liquid resulting from step (I)(a) is filtered either before or after addition of the polar organic solvent in step (II)(a).
15) In another embodiment the disclosure relates to the process of any one of embodiments 1) to 13), wherein the liquid resulting from step (I)(a) is first filtered before treatment with the non-ionic adsorbent in step (II′)(a).
16) In another embodiment the disclosure relates to the process of any one of embodiments 1) to 11), wherein the process comprises step (I)(b).
17 In another embodiment the disclosure relates to the process of any one of embodiments 1) to 14) and 16), wherein the process comprises steps (II)(a) and (II)(b) and optionally step (II)(c).
18) In another embodiment the disclosure relates to the process of any one of embodiments 1) to 14) and 16) to 17), wherein the polar organic solvent used in step (II)(a) is an alcohol, or a mixture of different alcohols, preferably containing 1-6 carbon atoms in linear or branched form.
19) In another embodiment the disclosure relates to the process of embodiment 18), wherein the alcohol is ethanol or propanol (especially 2-propanol), preferably ethanol.
20) In another embodiment the disclosure relates to the process of any one of embodiments 1) to 14) and 16) to 19), wherein the cation exchange adsorbent used in step (II)(b) is a strong acid cation exchange adsorbent, especially a strong acid cation exchange resin, preferably in H-Form and preferably a macroporous resin.
21) In another embodiment the disclosure relates to the process of any one of embodiments 1) to 14) and 16) to 20), wherein the weak base anion exchange adsorbent used in step (II)(b) is a weak base anion exchange resin, especially a weak base anion exchange macroporous resin.
22) In another embodiment the disclosure relates to the process of any one of embodiments 1) to 14) and 16) to 21), wherein the weak base anion exchange adsorbent used in step (II)(b) is converted to the acetate form before being used.
23) In another embodiment the disclosure relates to the process of any one of embodiments 1) to 14) and 16) to 22), wherein the process comprises step (II)(b)(i).
24) In another embodiment the disclosure relates to the process of any one of embodiments 1) to 14) and 16) to 22), wherein the process comprises step (II)(b)(ii).
25) In another embodiment the disclosure relates to the process of any one of embodiments 1) to 14) and 16) to 24), wherein step (II)(c) is part of the process.
26) In another embodiment the disclosure relates to the process of embodiment 25), wherein the adsorbent used in step (II)(c) is carbon, especially activated carbon, preferably granular activated carbon.
27) In another embodiment the disclosure relates to the process of any one of embodiments 1) to 13) and 15) to 16), wherein the process comprises steps (II′)(a) and (II′)(b) and optionally step (II′)(c).
28) In another embodiment the disclosure relates to the process of embodiment 27), wherein the non-ionic adsorbent used in step (II′)(a) is a non-ionic resin, especially a macroporous non-ionic resin, and preferably a hydrophobic resin.
29) In another embodiment the disclosure relates to the process of embodiment 27) or 28), wherein desorption of the bound vanillin into a solution in step (II′)(a)(ii) is carried out using an organic solvent, a mixture of different organic solvents, a mixture of water and an organic solvent, or a mixture of water and different organic solvents as eluent.
30) In another embodiment the disclosure relates to the process of embodiment 29), wherein the eluent is an alcohol, or a mixture of different alcohols, preferably containing 1-6 carbon atoms in linear or branched form.
31) In another embodiment the disclosure relates to the process of embodiment 30), wherein the alcohol is ethanol or propanol (especially 2-propanol), preferably ethanol.
32) In another embodiment the disclosure relates to the process of any one of embodiments 27) to 31), wherein the cation exchange adsorbent used in step (II′)(b) is a strong acid cation exchange adsorbent, especially a strong acid cation exchange resin, preferably in H-Form and preferably a macroporous resin.
33) In another embodiment the disclosure relates to the process of any one of embodiments 27) to 32), wherein the weak base anion exchange adsorbent used in step (II′)(b) is a weak base anion exchange resin, especially a weak base anion exchange macroporous resin.
34) In another embodiment the disclosure relates to the process of any one of embodiments 27) to 33), wherein the weak base anion exchange adsorbent used in step (II′)(b) is converted to the acetate form before being used.
35) In another embodiment the disclosure relates to the process of any one of embodiments 27) to 32), wherein the process comprises step (II′)(b)(i).
36) In another embodiment the disclosure relates to the process of any one of embodiments 27) to 31) and 33) to 34), wherein the process comprises step (II′)(b)(ii).
37) In another embodiment the disclosure relates to the process of any one of embodiments 27) to 34), wherein the process comprises step (II′)(b)(iii).
38) In another embodiment the disclosure relates to the process of any one of embodiment 37), wherein in step (II′)(b)(iii) the vanillin solution is treated with a cation exchange adsorbent followed by a weak base anion exchange adsorbent.
39) In another embodiment the disclosure relates to the process of any one of embodiments 27) to 38), wherein step (II′)(c) is part of the process.
40) In another embodiment the disclosure relates to the process of embodiment 39), wherein the adsorbent used in step (II′)(c) is carbon, especially activated carbon, preferably granular activated carbon.
41) In another embodiment the disclosure relates to the process of any one of embodiments 1) to 40), wherein the process does not contain a purification step with a strong base anion exchange adsorbent.
42) In another embodiment the disclosure relates to the process of any one of embodiments 1) to 41), wherein the process occurs at a pH below 7, preferably below 6.
43) In another embodiment the disclosure relates to the process of any one of embodiments 1) to 42), wherein before performing the crystallization in step (III), the vanillin solution is polished by filtration to remove any traces of adsorbents.
44) In another embodiment the disclosure relates to the process of any one of embodiments 1) to 43), wherein the crystallization in step (III) comprises concentration of the obtained vanillin solution.
45) In another embodiment the disclosure relates to the process of any one of embodiments 1) to 44), wherein crystallization of vanillin is performed at a pH of about 3.5-5.5, preferably about 4-5.5.

### EXAMPLES

Abbreviations as used herein:
BV bed volume(s)
DSP downstream process
GC gas chromatography
h hour(s)
HPLC high performance liquid chromatography
UF ultrafiltration
VAN vanillin
v/v volume/volume
w/w weight/weight

### Example 1

### Downstream Process A

Fermentation broth, produced by a vanillin glucoside-producing yeast strain that is capable of producing and secreting vanillin glucoside, was used to test the full downstream process type A. The vanillin glucoside-producing yeast strain used in the present and the following Examples is a Saccharomyces cerevisiae strain that has been engineered to comprise the de novo synthetic pathway as described for example in Hansen et al., Appl. Environ. Microbiol. 75 (9): 2765-2774 (2009) allowing production and secretion of vanillin glucoside. The fermentation broths used in the present and the following Examples contained vanillin glucoside at concentrations of >10 g/L. The fermentation broth was centrifugated and the obtained supernatant filtered with addition of Dal-Cin Alfatex 101 as filter aid. The resulting 4160 mL of filtered solution contained vanillin glucoside and had a pH 4.86. 0.46 g of the β-glucosidase solution from Biocatalysts Beta Glucosidase G016L (β-glucosidase activity: 10 u/g, biological source: Trichoderma longibrachiatum, enzyme concentration: approx. 6.3%) per 1 g of vanillin glucoside were added to the solution and the solution was stirred at 50° C. for 48 h. The formed slurry was filtered by addition of Dal-Cin Alfatex 101 as filter aid and diluted with ethanol to 20% v/v ethanol in water. Two lab scale columns were connected in series: a lab scale column filled with 300 mL of strong acid cation exchange Purolite C150SH resin (regenerated and activated with H₂SO₄ 5%) followed by a lab scale column filled with 300 ml of weak base anion exchange Purolite A845S resin (treated with acetic acid 5%). Then 3090 mL of the vanillin in aqueous ethanol 20% v/v solution with pH 5.04 and conductivity of 7.28 μS/cm were pumped in upflow direction at a flow rate of 4.8 BV/h through the two columns starting with the strong acid cation exchange resin. Afterwards, the resins were rinsed with aqueous ethanol 20% v/v. The following fractions were collected and analyzed:

| | Volume | | Conductivity | Color |
|---|---|---|---|---|
| Fraction | (mL) | pH | (μS/cm) | (sight) |
| Fraction 1 | 1000 | 3.48 | 96 | yellow |
| Fraction 2 | 1000 | 3.73 | 430 | brown |
| Fraction 3 | 1000 | 4.31 | 2383 | dark brown |
| Rinse | 700 | 4.63 | 3 | brown |

1.75 g of chemical granular activated carbon Chemviron Acticarbone BGE per g of vanillin was added to the merged three main fractions obtained in the previous step. The brown solution was stirred at room temperature for 3 h and then filtered and a yellow solution was obtained after carbon treatment. Carbon was rinsed with 600 mL of ethanol under stirring at room temperature for 1 h. Samples of each solution were filtered and analyzed. Vanillin yield of the carbon treatment was 81.89%.

Solutions treated with active carbon and the rinse fraction were merged and filtered over 0.45 μm Nylon filter followed by concentration under vacuum by rotary evaporator in a water bath at 55° C. up to 750 mL. The concentrate was cooled down to room temperature and the crystallization occurred. After stirring the slurry at room temperature overnight, formed solids were separated by vacuum filtration, rinsed with 300 mL of pre-cooled demineralized water and dried at 45° C. under vacuum for 3 h. Off-white-to-yellowish vanillin with purity of 97.49% w/w by GC and of 97.45% w/w by HPLC was obtained.

### Example 2

Downstream Process A, without Activated Carbon Treatment, Crystallization at Different Values of pH

3465 mL of fermentation broth with pH 4.75 comprising vanillin glucoside, produced by a vanillin glucoside-producing yeast strain, were used for the following experiment. Fermentation broth was centrifuged and the obtained supernatant filtered with Dal-Cin Alfatex 101 as filter aid. 2 L of the resulting solution, which had a pH of 4.83, were used for the enzymatic hydrolysis of vanillin glucoside. 0.26 g of the β-glucosidase solution from Biocatalysts Beta Glucosidase G016L (β-glucosidase activity: 10 u/g, biological source: Trichoderma longibrachiatum, enzyme concentration: approx. 6.3%) per 1 g of vanillin glucoside were added to the solution and the solution was stirred at 50° C. for 48 h. The formed slurry was filtered by addition of Dal-Cin Alfatex 101 as filter aid and diluted with ethanol to 50% v/v ethanol in water. Two lab scale columns were connected in series: a lab scale column filled with 200 mL of strong acid cation exchange resin Dowex 50 W×4 200-400 mesh (regenerated and activated with H₂SO₄) followed by a lab scale column filled with 200 ml of weak base anion exchange resin DuPont Amberlite FPA 53 (treated with acetic acid 5%). Then 3.15 L of vanillin solution, obtained in the previous step, were pumped in upflow direction at a flow rate of 4 BV/h through two columns starting with the strong acid cation exchange resin. Afterwards, the resins were rinsed with aqueous ethanol 50% v/v. The following three fractions were collected and analyzed:

| | Volume | | Conductivity | Color |
|---|---|---|---|---|
| Fraction | (mL) | pH | (μS/cm) | (sight) |
| Feed | 3150 | 4.80 | 14 | brown |
| Fraction 1 | 2000 | 3.83 | 54 | yellow |
| Fraction 2 | 1300 | 4.66 | 916 | brown |
| Rinse | 400 | 5.33 | 2392 | dark brown |

The first fraction with pH 3.83 was used to test the crystallization of vanillin at different values of pH.
a) The pH of 500 mL of the “Fraction 1” was adjusted from pH 3.83 to 5.06 with NaOH 30%. During the pH adjustment, the solution's color did not change. Then the solution was concentrated under vacuum by rotary evaporator in a water bath at 55° C. and black oil spots were observed in it. After cooling down to room temperature, pure ethanol was added until the black spots were completely dissolved. Then seeding was done with 30.34 mg of vanillin powder with purity ≥98% and the solution was kept at 4° C. for 24 h. Afterwards, formed solids were separated from mother liquor by vacuum filtration, rinsed with 10 mL of pre-cooled water and dried at 45° C. under vacuum for 3 h. Vanillin yield of the crystallization, filtration, wet solids rinsing, and drying was 88.47%. Brownish crystalline vanillin with purity of 98.62% w/w by HPLC was obtained.
b) The pH of 500 mL of the “Fraction 1” was adjusted from pH 3.83 to 7.20 with NaOH 30%. During the pH adjustment, the color of the solution changed from yellow to orange. The solution was concentrated under vacuum by rotary evaporator in a water bath at 55° C. and black oil spots were observed in it. After cooling down to room temperature, pure ethanol was added until the black spots were completely dissolved. Then seeding was done with 34.02 mg of vanillin with a purity ≥98% and the solution was kept at 4° C. for 3 days. Formed solids were separated from mother liquor by vacuum filtration, rinsed with 10 mL of pre-cooled water and dried at 45° C. under vacuum for 3 h. Vanillin yield of the crystallization, filtration, wet solids rinsing, and drying was 73.15%. Dark yellowish vanillin with purity of 99.14% w/w by HPLC was obtained.
c) The pH of 500 mL of the “Fraction 1” was adjusted from pH 3.83 to 8.57 with NaOH 30%. During the pH adjustment, the color of the solution became dark red and afterwards turned into dark brown. The solution was concentrated under vacuum by rotary evaporator in a water bath at 55° C. and after cooling down to room temperature, it was kept at 4° C. overnight. Formed solids were separated from the mother liquor by vacuum filtration, rinsed with 10 mL of pre-cooled water and dried at 45° C. under vacuum for 3 h. Vanillin yield of the crystallization, filtration, wet solids rinsing, and drying was 57.60%. Dark brown vanillin with purity of 97.39% w/w by HPLC was obtained.

### Example 3

### Downstream Process A by Using 2-Propanol as Solvent

17.7 L of fermentation broth with pH 4.96 comprising vanillin glucoside, produced by a vanillin glucoside-producing yeast strain, were used for the following experiment. Fermentation broth was ultrafiltered by the Alfa Laval TestUnit M20 with the UF 1 kDa Alfa Laval ETNA01PP membranes, diafiltered and concentrated to 4.90 L by reverse osmosis with the Alfa Laval RO98pHt membrane.

0.27 g of the β-glucosidase solution from Biocatalysts Beta Glucosidase G016L (β-glucosidase activity: 10 u/g, biological source: Trichoderma longibrachiatum, enzyme concentration: approx. 6.3%) per 1 g of vanillin glucoside were added to 4900 mL of the concentrated vanillin glucoside solution (pH 5.23) and the solution was stirred at 50° C. for 120 h.

The formed slurry was filtered in the presence of Dal-Cin Alfatex 101 as filter aid and diluted with 2-propanol up to a final concentration of 40% 2-propanol v/v. The obtained solution had pH 5.80. Two lab scale columns were connected in series: a lab scale column filled with 300 mL of strong acid cation exchange resin Purolite C150SH (activated and regenerated with H₂SO₄ 5%) followed by a lab scale column filled with 300 mL of weak base anion exchange resin Purolite A845S (treated with acetic acid 5%). Then 900 mL of vanillin in aqueous 2-propanol 40% v/v solution were pumped in upflow direction at a flow rate of 4 BV/h through the two columns starting with the strong acid cation exchange resin.

Afterwards, the resins were rinsed with aqueous 2-propanol 40% v/v. Four fractions were collected and analyzed:

| | Volume | | Conductivity | Color |
|---|---|---|---|---|
| Fraction | (mL) | pH | (μS/cm) | (sight) |
| Fraction 1 | 300 | 4.13 | 79 | red |
| Fraction 2 | 300 | 4.33 | 178 | dark brown |
| Fraction 3 | 300 | 4.52 | 420 | dark brown |
| Rinse | 600 | 4.52 | 570 | dark red |

The three main fractions and the rinse fraction, obtained in the previous step, were merged. Chemical granular activated carbon Chemviron Acticarbone BGE (0.59 kg BGE/1 kg VAN) was added to the obtained solution and it was stirred at room temperature for 3 h in batch. No significant color reduction of the solution was observed. Then carbon was filtered off and rinsed in batch with 133 mL of 40% v/v 2-propanol in water under stirring at room temperature for 1 h. The main solution after carbon treatment was merged with the carbon rinse fraction, filtered over 0.45 μm Nylon filter, and concentrated under vacuum by rotary evaporator in a water bath at 55° C. up to 90 mL. The concentrate was cooled down to room temperature under continuous stirring and then it was kept at 4° C. overnight. Seeding was performed by adding 40.99 mg of vanillin with a purity of 99% and the crystallization occurred immediately. Formed solids were separated from the mother liquor by vacuum filtration, rinsed with 40 mL of pre-cooled demineralized water and dried at 45° C. under vacuum for 3 h. Yellowish vanillin with purity of 92.75% w/w by HPLC and 96.18% w/w by GC was obtained.

### Example 4

Downstream Process A without Activated Carbon Treatment

3465 mL of fermentation broth with pH 4.75 comprising vanillin glucoside, produced by a vanillin glucoside-producing yeast strain, were used for the following experiment. Fermentation broth was centrifuged and the obtained supernatant filtered with Dal-Cin Alfatex 101 as filter aid.

0.26 g of the β-glucosidase solution from Biocatalysts Beta Glucosidase G016L (β-glucosidase activity: 10 u/g, biological source: Trichoderma longibrachiatum, enzyme concentration: approx. 6.3%) per 1 g of vanillin glucoside were added to 2 L of the filtered supernatant (pH 4.83) and the solution was stirred at 50° C. for 48 h. The formed slurry was filtered by addition of Dal-Cin Alfatex 101 as filter aid and diluted with ethanol to form 50% v/v ethanol in water.

A lab scale column was filled with 200 mL of strong acid cation exchange resin Dowex 50 W×4 200-400 mesh (activated with H₂SO₄) and 200 mL of the filtered solution with ethanol was pumped through the column in upflow direction at a flow rate of 1.5 BV/h. Afterwards, the resin was rinsed with aqueous ethanol 50% v/v and three different fractions were collected and analyzed:

| | Volume | | Conductivity | Color |
|---|---|---|---|---|
| Fraction | (mL) | pH | (μS/cm) | (sight) |
| Feed | 200 | 4.80 | 14 | brown |
| Main fraction | 200 | 2.18 | 25 | orange |
| Rinse fraction 1 | 200 | 2.51 | 13 | orange |
| Rinse fraction 2 | 200 | 3.68 | 53 | yellow |

A lab scale column was filled with 200 ml of weak base anion exchange resin DuPont Amberlite FPA 53 (treated with 5% acetic acid) and the three fractions obtained in the previous step were pumped separately, starting with the main fraction, through the resin column in upflow direction at a flow rate of 7.2 BV/h. Afterwards, the resin was rinsed with aqueous ethanol 50% v/v and four different fractions were collected and analyzed:

| | Volume | | Conductivity | Color |
|---|---|---|---|---|
| Fraction | (mL) | pH | (μS/cm) | (sight) |
| Main fraction | 190 | 3.41 | 33 | pale yellow |
| Fraction 1 | 200 | 3.41 | 42 | yellow |
| Fraction 2 | 190 | 3.84 | 40 | yellow |
| Fraction 3 | 214 | 4.02 | 28 | light yellow |

The solution obtained by merging all the fractions obtained in the previous step was pumped through a 0.22 μm PTFE filter and 800 mL of the clear filtrate was concentrated under vacuum by rotary evaporator in a water bath at 55° C. up to 52 mL. The concentrate was cooled down to room temperature and seeding was done with 24 mg of vanillin with a purity ≥98%. The crystallization occurred immediately, and the solution was kept at 4° C. overnight. Formed solids were separated from the mother liquor by vacuum filtration, rinsed with 10 mL of pre-cooled demineralized water and dried at 45° C. under vacuum for 3 h. Yellow vanillin with purity of 96.5% w/w by HPLC and 98.5% w/w by GC was obtained.

### Example 5

Adsorption and Release of Vanillin onto/Off the Strong Base Anion Exchange Resin after Treatment With Strong Acid Cation Exchange Resin and Weak Base Anion Exchange Resin

9.58 L of fermentation broth with pH 5.01 comprising vanillin glucoside, produced by a vanillin glucoside-producing yeast strain, were used for the following experiment. The broth was ultrafiltered by the Alfa Laval TestUnit M20 with the UF 1 kDa Alfa Laval ETNA01PP membranes, diafiltered and concentrated by reverse osmosis with the Alfa Laval RO98pHt membranes.

The resulting 4950 mL of concentrated vanillin glucoside solution had a pH of 5.03. 0.26 g of the β-glucosidase solution from Biocatalysts Beta Glucosidase G016L (β-glucosidase activity: 10 u/g, biological source: Trichoderma longibrachiatum, enzyme concentration: approx. 6.3%) per 1 g of vanillin glucoside were added to the solution and the solution was stirred at 50° C. for 48 h. The formed slurry was filtered with addition of Dal-Cin Alfatex 101 as filter aid, followed by dilution with ethanol to form a final concentration of 20% ethanol v/v. The pH of the obtained solution was 5.34.

Two lab scale columns were connected in series: a lab scale column of 350 mL filled with 300 mL of strong acid cation exchange resin Purolite C150SH (activated and regenerated with H₂SO₄ 5%) followed by a lab scale column of 350 mL filled with 300 ml of weak base anion exchange resin Purolite A845S (treated with acetic acid 5%), and 1.17 L of clear filtrate with ethanol were pumped through the two resin columns in upflow direction at a flow rate of 4 BV/h, starting with the strong acid cation exchange resin. Afterwards both resin columns were rinsed with aqueous ethanol 20% v/v. Five different fractions were collected and analyzed:

| | Volume | | Conductivity | Color |
|---|---|---|---|---|
| Fraction | (mL) | pH | (μS/cm) | (sight) |
| Feed | 1170 | 5.49 | 10 | dark brown |
| Fraction 1 | 585 | 3.72 | 361 | brown |
| Fraction 2 | 585 | 4.00 | 1378 | dark brown |
| Rinse fraction 1 | 300 | 4.27 | 2259 | brown |
| Rinse fraction 2 | 300 | 4.03 | 657 | orange |
| Rinse fraction 3 | 300 | 3.94 | 336 | yellow |

To a lab scale column with 45 mL of strong base anion exchange resin DuPont Amberlite FPA 42 (activated with NaOH 5% and rinsed with demineralized water) was delivered 225 ml of the solution obtained by merging all the above fractions. The merged solution was pumped through the column at a flow rate of 4 BV/h until five fractions were collected and afterwards the resin is rinsed with demineralized water until three rinse fractions were collected and analyzed:

| Fraction | Volume (mL) | Color (sight) |
|---|---|---|
| Fraction 1 | 45 | yellow |
| Fraction 2 | 45 | orange |
| Fraction 3 | 45 | dark orange |
| Fraction 4 | 45 | brown |
| Fraction 5 | 45 | dark orange |
| Rinse fraction 1 | 45 | orange |
| Rinse fraction 2 | 45 | yellow |
| Rinse fraction 3 | 45 | yellow |

Afterwards vanillin was released by pumping aqueous ethanol 70% v/v with 5% acetic acid at a flow rate of 4 BV/h through the resin column. Five fractions during the release of vanillin were collected and analyzed.

| Fraction | Volume (mL) | Color (sight) |
|---|---|---|
| Release fraction 1 | 45 | yellow |
| Release fraction 2 | 45 | orange |
| Release fraction 3 | 45 | yellow |
| Release fraction 4 | 45 | yellow |
| Release fraction 5 | 45 | yellow |

67.86% of vanillin adsorbed onto the strong base anion exchange resin were released by elution with aqueous ethanol 70% v/v with 5% acetic acid, but the overall bound vanillin by Amberlite FPA42 was only 13.60% from the total quantity delivered by the feed solution. Thus, the overall vanillin yield of the purification by the strong base anion exchange resin was 9.22%. Due to the low efficiency resulting from the application of the strong base anion exchange resin, the purification process in this example was stopped without performing a crystallization.

### Example 6

### Downstream Process B

17.7 L of fermentation broth with pH 4.96 comprising vanillin glucoside, produced by a vanillin glucoside-producing yeast strain, were used for the following experiment. The broth was ultrafiltered by the Alfa Laval TestUnit M20 with the UF 1 kDa Alfa Laval ETNA01PP membranes, diafiltered and concentrated up to 4.90 L by reverse osmosis with the Alfa Laval RO98pHt membranes.

0.27 g of the β-glucosidase solution from Biocatalysts Beta Glucosidase G016L (β-glucosidase activity: 10 u/g, biological source: Trichoderma longibrachiatum, enzyme concentration: approx. 6.3%) per 1 g of vanillin glucoside were added to 4900 mL of the concentrated vanillin glucoside solution (pH of 5.23) and the solution was stirred at 50° C. for 120 h. 850 mL of the formed slurry was filtered with addition of Dal-Cin Alfatex 101 as filter aid. The clear filtrate containing vanillin was pumped in downflow direction through a 200 mL column filled with non-ionic resin Purolite Purosorb PAD400 (pre-conditioned with demineralized water) at a flow rate of 4 BV/h. Five fractions of 500 mL (2.5 BV each) were collected (850 mL corresponded to the feed and 1650 ml to the rinse). After feed loading, the resin was rinsed with 8 BV of demineralized water to remove colored impurities until water was coming out of the resin column with a pale yellow shade. Thereafter, vanillin was released from the resin by eluting with aqueous 80% v/v ethanol in upflow direction at a flow rate of 4 BV/h. 1.672 L of eluent in eight fractions were collected. Vanillin desorbed quickly and the desorbate had a brown color while more diluted desorbate was yellowish.

Desorbate fractions from non-ionic resin Purolite Purosorb PAD400 were pumped through the strong acid cation exchange resin Dowex 50 W×4 in H-form (BV=200 mL), wherein the resin column was well prerinsed with demineralized water and preconditioned with 80% v/v ethanol in water. The desorbate fractions were pumped through the resin column one after the other, starting with the first one and ending with the last one, in downflow direction at a flow rate of 4 BV/h. At the end, 80% v/v ethanol in water was pumped through the column to rinse the resin. In total eight fractions of 200 mL were collected.

The fractions collected after the strong acid cation exchange resin were pumped through a column filled with weak base anion exchange resin DuPont Amberlite FPA53 (BV=200 mL) previously treated with 5% acetic acid, rinsed with demineralized water and preconditioned with 80% v/v ethanol in water. Fractions were pumped through the resin column one after the other starting with the first fraction of the previous step and ending with the last one in downflow direction at a flow rate of 4 BV/h. Then, 80% v/v ethanol in water was pumped through the column to rinse the resin. In total eight fractions of 200 mL were collected.

All the collected fractions were merged for carbon treatment and granular activated carbon Chemviron Acticarbone BGE (ratio 0.59 kg BGE/kg VAN) was loaded into a column of 50 mL and the orange solution of the merged fractions from the resin treatment was recirculated through the carbon column in downflow direction at a flow rate of 8 BV/h for 20 h. The color of the solution due to carbon treatment changed from orange to yellow. The carbon column was rinsed with 5 BV of 80% v/v ethanol in water (5×50 mL). The main fraction treated with carbon and the carbon rinse fractions were merged.

The obtained solution was filtered over 0.48 μm PTFE filter to remove residual particles of carbon and the filter was rinsed with 40 mL of 80% v/v ethanol in water, giving in total a volume of 1740 mL with pH 4.94 and a conductivity of 15 μS/cm.

The clear filtrate was concentrated under vacuum (from 220 mbar down to 80 mbar) by rotary evaporator in a water bath at 55° C. To the residual yellow concentrate 4 mL of ethanol 96% were added to initiate the crystallization. Seeding was performed with 20 mg of pure vanillin. Solution was cooled down and crystallization occurred at around 35° C. forming vanillin crystals. Slurry was stirred at room temperature for 16 h and afterwards it was kept at 4° C. for 4 h, allowing crystals to mature.

Vanillin crystals were filtered off on a Büchner funnel, rinsed with pre-cooled demineralized water (100 mL) and dried under vacuum (10 mbar, 50° C.) for 16 h. Light yellow crystalline vanillin with purity of 99.44% w/w by HPLC and 99.28% w/w by GC was obtained.

### Example 7

Downstream Process B without the Use of Strong Acid Cation Exchange Resin

17.7 L of fermentation broth with pH 4.96 comprising vanillin glucoside, produced by a vanillin glucoside-producing yeast strain, were used for the following experiment. The broth was ultrafiltered by the Alfa Laval TestUnit M20 with the UF 1 kDa Alfa Laval ETNA01PP membranes, then diafiltered and concentrated up to 4.90 L by reverse osmosis with the Alfa Laval RO98pHt membranes.

0.27 g of the β-glucosidase solution from Biocatalysts Beta Glucosidase G016L (β-glucosidase activity: 10 u/g, biological source: Trichoderma longibrachiatum, enzyme concentration: approx. 6.3%) per 1 g of vanillin glucoside were added to 4900 mL of the concentrated vanillin glucoside solution (pH 5.23) and the solution was stirred at 50° C. for 120 h. The formed slurry was filtered by pressure filter in the presence of Dal-Cin Filtex 7 as filter aid (25 g).

800 mL of clear filtrate was used as feed and 200 ml of water, used for rinsing the cake of filter aid, were used as well to feed and rinse the non-ionic resin in the following step. A 200 mL column filled with the non-ionic resin Purolite Purosorb PAD400 was rinsed with water. Feed was pumped through the column in downflow direction (filtrate, 800 mL) followed by rinse-feed (200 mL, prepared by rinsing the filter-aid) at a flow rate of 4 BV/h. Fractions of 200 ml (1 BV) were collected and the resin was additionally rinsed with 7 BV of water to remove colors and impurities until the eluted water had a pale yellow shade. Afterwards, vanillin was released from the resin by eluting with 80% v/v aqueous ethanol.

The desorbate from the non-ionic resin Purolite Purosorb PAD400 was delivered to the weak base anion exchange resin DuPont Amberlite FPA53 (BV=200 mL) previously treated with 5% acetic acid, rinsed with demineralized water, and preconditioned with 80% v/v ethanol in water. Fractions were pumped in downflow direction at a flow rate of 4 BV/h one after the other starting with the first fraction of the previous step and ending with the last one. Afterwards 80% v/v ethanol in water was pumped through the resin column in upflow direction at a flow rate of 4 BV/h. In total nine fractions of 200 mL were collected.

The first five fractions from the weak base anion exchange resin were merged for carbon treatment. Granular activated carbon Chemviron Acticarbone BGE (ratio 0.58 kg BGE/kg VAN) was loaded into a column of 50 mL and the red solution of the merged fractions was recirculated through the carbon column in downflow direction at a flow rate of 8 BV/h for 20 h. Solution color changed from red to orange. The carbon column was rinsed with 5 BV of 80% v/v ethanol in water (5×50 mL).

The main fraction after carbon treatment was pooled with five carbon rinse fractions and the combined solution was filtered over 0.48 μm PTFE filter to remove residual particles of carbon.

The filter was rinsed with 35 mL of 80% v/v ethanol in water, giving in total a volume of solution of 1285 mL, with pH 5.42 and conductivity of 52 μS/cm.

1285 mL of solution obtained in the previous step were concentrated under vacuum (from 220 mbar down to 80 mbar) by rotary evaporator in a water bath at 55° C. 4 mL of ethanol 96% in water v/v were added to the residual yellow concentrate to initiate crystallization of vanillin. Seeding was performed by adding 20 mg of pure vanillin. Solution was cooled down and crystallization occurred at around 35° C. forming vanillin crystals. The slurry was stirred at room temperature for 16 h and afterwards it was kept at 4° C. for 4 h, allowing crystals to mature. Vanillin crystals were filtered off on a Büchner funnel, rinsed with pre-cooled demineralized water (100 mL) and dried in a vacuum oven (10 mbar, 50° C.) for 16 h. Light yellow crystalline vanillin with purity of 98.54% w/w by HPLC and 98.12% w/w by GC was obtained.

The flavor of vanillin obtained by the purification process of this Example 7, i.e., without the strong acid cation exchange resin treatment, was more off-note and stronger creamy versus the vanillin obtained in Example 6, which included the step with strong acid cation exchange resin. The flavor of vanillin obtained in Example 6 was more intense vanilla with a slight creamy note. The desired flavor is more intense vanilla and therefore the flavor of vanillin obtained in Example 6 is preferred.

### Example 8

Downstream Process B by Using 2-Propanol as Solvent 17.7 L of fermentation broth with pH 4.96 comprising vanillin glucoside, produced by a vanillin glucoside-producing yeast strain, were used for the following experiment. The broth was ultrafiltered by the Alfa Laval TestUnit M20 with the UF 1 kDa Alfa Laval ETNA01PP membranes, then diafiltered and concentrated to 4.90 L by reverse osmosis with the Alfa Laval RO98pHt membranes.

0.27 g of the β-glucosidase solution from Biocatalysts Beta Glucosidase G016L (β-glucosidase activity: 10 u/g, biological source: Trichoderma longibrachiatum, enzyme concentration: approx. 6.3%) per 1 g of vanillin glucoside were added to 4900 mL of the concentrated vanillin glucoside solution (pH 5.23) and the solution was stirred at 50° C. for 120 h. The obtained slurry was filtered with Dal-Cin Alfatex 101 as filter aid followed by dilution with ethanol to a final concentration of 20% ethanol v/v.

650 mL of the filtered vanillin solution were pumped in upflow direction at a flow rate of 4 BV/h through a lab scale column filled with 500 ml of non-ionic resin Purolite Purosorb PAD 400. Afterwards, resin was rinsed with demineralized water.

Vanillin was released from the resin by pumping aqueous 80% v/v 2-propanol in downflow direction at a flow rate of 4 BV/h through the resin column and six fractions of 500 mL were collected.

The first three release fractions collected from the non-ionic resin Purolite Purosorb PAD 400 were pumped in upflow direction at a flow rate of 4 BV/h through a column filled with 200 mL strong acid cation exchange resin Purolite C150SH (activated with H₂SO₄ 5%, rinsed with demineralized water and preconditioned with 80% v/v 2-propanol in water). Afterwards the resin was rinsed with aqueous 2-propanol 80% v/v. Ten fractions were collected and analyzed:

| | Volume | | Conductivity | Color |
|---|---|---|---|---|
| Fraction | (mL) | pH | (μS/cm) | (sight) |
| Feed | 1500 | 4.73 | 47 | dark brown |
| Fraction 1 | 200 | 3.50 | 39 | dark red |
| Fraction 2 | 200 | 3.36 | 70 | dark brown |
| Fraction 3 | 200 | 3.46 | 60 | dark brown |
| Fraction 4 | 200 | 3.44 | 54 | dark brown |
| Fraction 5 | 200 | 3.56 | 55 | dark brown |
| Fraction 6 | 200 | 3.61 | 51 | dark brown |
| Fraction 7 | 200 | 3.47 | 60 | dark brown |
| Fraction 8 | 200 | 3.54 | 37 | dark brown |
| Fraction 9 | 200 | 4.1 | 9 | orange |
| Fraction 10 | 200 | 4.34 | 5 | yellow |

The first eight fractions collected from the strong acid cation exchange resin, in the previous step, were pumped in downflow direction at a flow rate of 4 BV/h through a column filled with 200 ml of weak base anion exchange resin Purolite A845S previously treated with acetic acid 5%, rinsed with demineralized water and preconditioned with 80% v/v 2-propanol in water. Afterwards the resin was rinsed with aqueous 2-propanol 80% v/v. Ten fractions were collected and analyzed:

| | Volume | | Conductivity | Color |
|---|---|---|---|---|
| Fraction | (mL) | pH | (μS/cm) | (sight) |
| Fraction 1 | 200 | 5.05 | 4 | orange |
| Fraction 2 | 200 | 4.83 | 6 | red |
| Fraction 3 | 200 | 4.88 | 8 | red |
| Fraction 4 | 200 | 4.98 | 9 | red |
| Fraction 5 | 200 | 4.97 | 9 | red |
| Fraction 6 | 200 | 4.91 | 10 | red |
| Fraction 7 | 200 | 4.82 | 9 | red |
| Fraction 8 | 200 | 4.96 | 6 | orange |
| Fraction 9 | 200 | 5.15 | 3 | yellow |
| Fraction 10 | 200 | 5.32 | 2 | pale yellow |

Ten fractions collected from the weak base anion exchange resin in the previous step, were combined and the obtained dark red solution was pumped in downflow direction through a column filled with 50 mL of chemical granular activated carbon Chemviron Acticarbone BGE (ratio 0.59 kg BGE/kg VAN) in a recirculation loop at a flow rate of 8 BV/h at room temperature for three days. The solution after carbon treatment was orange. Carbon was rinsed with 5 BV of 80% v/v 2-propanol in water. Samples of each solution were filtered and analyzed. Decolorized main vanillin solution and the rinse fraction were merged and filtered over 0.45 μm Nylon filter.

The filtered and decolorized solution was concentrated under vacuum by rotary evaporator in a water bath at 55° C. up to 118 mL, and then cooled down to room temperature under continuous stirring. 4 mL of ethanol 100% were added to concentrate to solubilize the brown oil formed in the solution during the concentration and seeding was performed by adding 29.21 mg of vanillin (purity 99%). The solution was kept at 4° C. overnight. Formed solids were separated from the mother liquor by vacuum filtration, rinsed with 100 ml of pre-cooled demineralized water and dried at 45° C. under vacuum for 3 h. Dark yellow vanillin with purity of 97.77% w/w by HPLC and 99.70% w/w by GC was obtained.

### Example 9

Downstream Process B Wherein Weak Base Anion Exchange Resin Treatment Occurs Before the Treatment with Strong Acid Cation Exchange Resin

Fermentation broth containing vanillin glucoside was ultrafiltered and diafiltered over UF 10 kDa Alfa Laval ETNA10PP membranes, then concentrated with reverse osmosis over Alfa Laval RO99 membranes. The vanillin glucoside in the obtained concentrate was hydrolyzed with β-glucosidase solution from Biocatalysts Beta Glucosidase G016L (β-glucosidase activity: 10 u/g, biological source: Trichoderma longibrachiatum, enzyme concentration: approx. 6.3%) applying 0.45 kg of the enzyme solution per kg of vanillin glucoside at 55° C. for 30 h. The obtained suspension was filtered on filter press in the presence of the filter aid Dal-Cin Cellulose M09 to obtain a clear brown vanillin containing filtrate with a pH of 5.04. 1 L of this filtered vanillin solution was pumped in downflow direction at a flow rate of 4 BV/h through a column filled with 500 ml of non-ionic resin Purolite Purosorb PAD 400. Thereafter the resin was rinsed with 3 BV of demineralized water and vanillin was released from the resin by pumping aqueous 80% v/v ethanol in upflow direction at a flow rate of 4 BV/h through the resin column. Four BV were collected and merged.

1 L of the desorbate obtained in the previous step was pumped in downflow direction at a flow rate of 4 BV/h through a column filled with 200 ml of weak base anion exchange resin Purolite A845S pretreated with acetic acid 5%, rinsed with demineralized water and preconditioned with 80% v/v ethanol in water. Afterwards the resin was rinsed with aqueous ethanol 80% v/v. Seven fractions were collected and analyzed:

| | Volume | | Conductivity | Color |
|---|---|---|---|---|
| Fraction | (mL) | pH | (μS/cm) | (sight) |
| Feed | 1000 | 5.21 | 30 | dark brown |
| Fraction 1 | 200 | 5.38 | 115 | dark brown |
| Fraction 2 | 200 | 5.28 | 84 | dark brown |
| Fraction 3 | 200 | 5.25 | 72 | dark brown |
| Fraction 4 | 200 | 5.33 | 58 | dark brown |
| Fraction 5 | 200 | 5.39 | 47 | red |
| Fraction 6 | 200 | 5.16 | 22 | orange |
| Fraction 7 | 200 | 5.06 | 5 | yellow |

Seven fractions, obtained in the previous step, were pumped in downflow direction at a flow rate of 4 BV/h through a column filled with 200 ml of strong acid cation exchange resin Purolite C150SH (activated with H₂SO₄ 5%, rinsed with water and preconditioned with 80% v/v ethanol in water). Afterwards the resin was washed with aqueous ethanol 80% v/v. Seven fractions were collected and analyzed:

| | Volume | | Conductivity | Color |
|---|---|---|---|---|
| Fraction | (mL) | pH | (μS/cm) | (sight) |
| Fraction 1 | 200 | 4.27 | 13 | dark red |
| Fraction 2 | 200 | 4.02 | 23 | dark red |
| Fraction 3 | 200 | 4.05 | 21 | red |
| Fraction 4 | 200 | 4.14 | 16 | orange |
| Fraction 5 | 200 | 4.20 | 13 | orange |
| Fraction 6 | 200 | 4.30 | 11 | orange |
| Fraction 7 | 200 | 4.49 | 7 | yellow |

Seven fractions collected from the strong acid cation exchange resin were merged and the obtained dark red solution was recirculated in downflow direction through a column filled with chemical granular activated carbon from Chemviron Acticarbone BGE (ratio 0.48 kg BGE/kg VAN) at a flow rate of 8 BV/h at room temperature overnight. The solution was yellow at the end of the carbon treatment. Then the carbon was rinsed with 4 BV of 80% ethanol. Decolorized vanillin solution and the rinse fractions were merged, filtered over 0.45 μm Nylon filter and analyzed.

The filtered and decolorized solution, obtained in the previous step, was concentrated under vacuum by rotary evaporator in a water bath at 55° C. up to 135 mL and cooled down to room temperature under continuous stirring. 4 mL of ethanol 99.9% were added to this solution to solubilize the brown oil formed in the solution during the concentration. Afterwards seeding was performed by adding 35 mg of vanillin of purity ≥98%. The obtained slurry was stirred at room temperature overnight and afterwards solids were separated from the mother liquor by vacuum filtration, rinsed with 100 mL of pre-cooled demineralized water and dried at 50° C. under vacuum overnight. Yellow vanillin with purity of 99.60% w/w by GC was obtained.

### Example 10

Downstream Process B without Use of Weak Base Anion Exchange Resin and without Activated Carbon Treatment

Fermentation broth containing vanillin glucoside was ultrafiltered and diafiltered over UF 10 kDa Alfa Laval ETNA10PP membranes, then concentrated with reverse osmosis over Alfa Laval RO99 membranes. Vanillin glucoside in the obtained concentrate was hydrolyzed with β-glucosidase solution from Biocatalysts Beta Glucosidase G016L (β-glucosidase activity: 10 u/g, biological source: Trichoderma longibrachiatum, enzyme concentration: approx. 6.3%) applying 0.45 kg of the enzyme solution per kg of vanillin glucoside at 55° C. for 30 h. The obtained suspension was filtered on filter press in the presence of the filter aid Dal-Cin Cellulose M09 to obtain a clear brown vanillin containing filtrate with a pH of 5.04. 1 L of this filtered vanillin solution was pumped in downflow direction at a flow rate of 4 BV/h through a column filled with 500 mL of non-ionic resin Purolite Purosorb PAD 400. Afterwards the resin was rinsed with 3 BV of demineralized water. Vanillin was released from the resin by pumping aqueous 80% v/v ethanol in upflow direction at a flow rate of 4 BV/h through the resin column. Four BV were collected and analyzed.

700 mL of the desorbate, obtained in the previous step after the adsorption and release onto/off the non-ionic resin Purolite Purosorb PAD 400, were pumped in downflow direction through a column filled with 200 ml of strong acid cation exchange resin Purolite C150SH (activated with H₂SO₄ 5%), rinsed with water and preconditioned with 80% v/v ethanol in water at a flow rate of 4 BV/h. Afterwards, the resin was rinsed with aqueous ethanol 80% v/v. Five fractions were collected and analyzed:

| | Volume | | Conductivity | Color |
|---|---|---|---|---|
| Fraction | (mL) | pH | (μS/cm) | (sight) |
| Feed | 700 | 5.21 | 30 | dark brown |
| Fraction 1 | 200 | 3.44 | 102 | brown |
| Fraction 2 | 200 | 3.73 | 32 | brown |
| Fraction 3 | 200 | 3.73 | 33 | red |
| Fraction 4 | 200 | 3.76 | 30 | red |
| Fraction 5 | 200 | 4.19 | 12 | orange |

Five fractions, collected from the strong acid cation exchange resin in the previous step, were merged. The obtained solution was concentrated under vacuum by rotary evaporator in a water bath at 55° C. and cooled down to room temperature under continuous stirring. 4 mL of ethanol 99.9% were added to this solution to solubilize the brown oil formed in the solution during the concentration and afterwards seeding was performed by adding 35.84 mg of vanillin of purity ≥98%. The obtained slurry was stirred at room temperature overnight and afterwards solids were separated from the mother liquor by vacuum filtration, rinsed with 100 ml of pre-cooled demineralized water and dried at 50° C. under vacuum overnight. Brownish vanillin with purity of 94.10% w/w by GC was obtained.

### Example 11

Downstream Process B without Activated Carbon Treatment

Fermentation broth containing vanillin glucoside was ultrafiltered and diafiltered over UF 10 kDa Alfa Laval ETNA10PP membranes, then concentrated with reverse osmosis over Alfa Laval RO99 membranes. The vanillin glucoside in the obtained concentrate was hydrolyzed with β-glucosidase solution from Biocatalysts Beta Glucosidase G016L (β-glucosidase activity: 10 u/g, biological source: Trichoderma longibrachiatum, enzyme concentration: approx. 6.3%) applying 0.45 kg of the enzyme solution per kg of vanillin glucoside at 55° C. for 30 h. The obtained suspension was filtered on filter press in the presence of the filter aid Dal-Cin Cellulose M09 to obtain a clear brown vanillin containing filtrate with a pH of 5.04. 1 L of this filtered vanillin solution was pumped in downflow direction at a flow rate of 4 BV/h through a column filled with 500 ml of non-ionic resin Purolite Purosorb PAD 400. Afterwards the resin was rinsed with 3 BV of demineralized water and vanillin was released from the resin by pumping aqueous 80% v/v ethanol in upflow direction at a flow rate of 4 BV/h through the resin column. Four BV were collected and analyzed.

1 L of the desorbate obtained in the previous step was pumped in downflow direction at a flow rate of 4 BV/h through two 200 mL resin columns in series: the first column filled with strong acid cation exchange resin Purolite C150SH (activated with H₂SO₄ 5%) followed by a column filled with weak base anion exchange resin Purolite A845S (pretreated with acetic acid 5%). Afterwards the columns were rinsed with aqueous ethanol 80% v/v. A single fraction of 7 BV was collected and analyzed:

| | Volume | | Conductivity | Color |
|---|---|---|---|---|
| Fraction | (mL) | pH | (μS/cm) | (sight) |
| Feed | 1000 | 4.91 | 31 | dark brown |
| Main fraction | 1400 | 4.40 | 15 | orange |

The solution obtained in the previous step was concentrated under vacuum by rotary evaporator in a water bath at 55° C. up to 115 mL and cooled down to room temperature under continuous stirring. 4 mL of ethanol 99.9% were added to the solution to solubilize the brown oil formed during the concentration and the solution was stirred at room temperature overnight. Afterwards, formed solids were separated from the mother liquor by vacuum filtration, rinsed with 100 ml of pre-cooled demineralized water and dried at 50° C. under vacuum overnight. Yellow vanillin with purity of 97.10% w/w by GC was obtained.

### Example 12

Process B without Use of Weak Base Anion Exchange Resin

Fermentation broth containing vanillin glucoside was ultrafiltered and diafiltered over UF 10 kDa Alfa Laval ETNA10PP membranes, then concentrated with reverse osmosis over Alfa Laval RO99 membranes. The vanillin glucoside in the obtained concentrate was hydrolyzed with β-glucosidase solution from Biocatalysts Beta Glucosidase G016L (β-glucosidase activity: 10 u/g, biological source: Trichoderma longibrachiatum, enzyme concentration: approx. 6.3%) applying 0.45 kg of the enzyme solution per kg of vanillin glucoside at 55° C. for 30 h. The obtained suspension was filtered over filter press in the presence of the filter aid Dal-Cin Cellulose M09 to obtain a clear brown vanillin containing filtrate with a pH of 5.04. 1 L of this filtered vanillin solution was pumped in downflow direction at a flow rate of 4 BV/h through a column filled with 500 mL of non-ionic resin Purolite Purosorb PAD 400. Afterwards the resin was rinsed with 3 BV of demineralized water and vanillin was released from the resin by pumping aqueous 80% v/v ethanol in upflow direction at a flow rate of 4 BV/h through the resin column. Four BV were collected and analyzed.

700 ml of the desorbate, obtained after the adsorption and release onto/off the non-ionic resin Purolite Purosorb PAD 400, were pumped in downflow direction at a flow rate of 4 BV/h through a column filled with 200 mL of strong acid cation exchange resin Purolite C150SH (activated with H₂SO₄ 5%), rinsed with water and preconditioned with 80% v/v ethanol in water. Afterwards the resin column was rinsed with aqueous ethanol 80% v/v. One fraction of 1 L was collected and analyzed:

| | Volume | | Conductivity | Color |
|---|---|---|---|---|
| Fraction | (mL) | pH | (μS/cm) | (sight) |
| Feed | 700 | 5.21 | 30 | dark brown |
| Main fraction | 1000 | 3.53 | 31 | brown |

The fraction collected after the strong acid cation exchange resin was recirculated in downflow direction through a column filled with granular activated carbon Chemviron Acticarbone BGE (ratio 0.53 kg BGE/kg VAN) at a flow rate of 8 BV/h at room temperature overnight. The solution was orange after carbon treatment. Carbon was rinsed with 4 BV of 80% v/v ethanol in water and the main solution is combined with the rinse fractions and filtered over 0.45 μm Nylon filter.

The clear filtrate was concentrated under vacuum by rotary evaporator in a water bath at 55° C. up to 240 mL and cooled down to room temperature under continuous stirring. 4 mL of ethanol 99.9% were added to this solution to solubilize the brown oil formed in the solution during the concentration and the resulting solution was stirred at room temperature overnight to form the slurry. Solids were separated from the mother liquor by vacuum filtration, rinsed with 100 ml of pre-cooled demineralized water and dried at 50° C. under vacuum overnight. Yellowish vanillin with purity of 95.90% w/w by GC was obtained.

### Example 13

### Downstream Process C

11.56 L of fermentation broth comprising vanillin glucoside, produced by a vanillin glucoside-producing yeast strain, were used to test the full downstream process type C. 0.57 g of the β-glucosidase solution from Biocatalysts Beta Glucosidase G016L (β-glucosidase activity: 10 u/g, biological source: Trichoderma longibrachiatum, enzyme concentration: approx. 6.3%) per 1 g of vanillin glucoside were added to the fermentation broth and stirred at 55° C. for 48 h.

The broth with pH 5.03 was ultrafiltered and diafiltered on Alfa Laval TestUnit M20 with the UF 10 kDa ETNA10PP membranes. 2.5 L of permeate were pumped through the non-ionic resin Purolite Purosorb PAD 400 (500 mL) in upflow direction at a flow rate of 4 BV/h. The resin column was rinsed with 3 BV of demineralized water and vanillin desorbed by pumping 2.5 BV of 80% v/v ethanol in water through the resin column followed by approx. 600 ml of water. Four desorbate fractions of 500 mL were collected.

Two columns were connected in series: a column filled with 200 ml of strong acid cation exchange resin Purolite C150SH H-form followed by a column filled with 200 ml of weak base anion exchange resin DuPont Amberlite FPA53 (pretreated with acetic acid 5%). The four fractions of desorbate from the previous step were merged and pumped in upflow direction through the two columns in series, starting with the strong acid cation exchange resin. Four fractions were collected and analyzed:

| | Volume | | Conductivity | Color |
|---|---|---|---|---|
| Fraction | (mL) | pH | (μS/cm) | (sight) |
| Fraction 1 | 500 | 5.11 | 151 | red |
| Fraction 2 | 500 | 5.14 | 60 | orange |
| Fraction 3 | 500 | 5.30 | 15 | yellow |
| Fraction 4 | 500 | 5.22 | 8 | yellow |

All four fractions were combined to give a solution that was pumped in downflow direction at a flow rate of 4 BV/h through a column filled with the granular activated carbon Chemviron Acticarbone BGE (ratio 0.50 kg BGE/kg VAN) for 16 h. The color of the solution changed from amber to yellow due to the carbon treatment. Afterwards, carbon was rinsed with 3 BV of 80% ethanol in water and the main solution treated with carbon was merged with the rinse fractions before proceeding to filtration over 0.48 μm PTFE filter.

The clear filtrate (pH 4.50) was concentrated at 60° C. under vacuum (300-150 mbar) in a 1 L reactor to give 280 mL of a biphasic emulsion (oily brown vanillin and water). The emulsion quickly turned into a slurry by cooling down to 20° C. and stirring for 16 h before it was additionally cooled down to 10-15° C. before separation of solids from the mother liquor by vacuum filtration. Wet solids were washed with 200 ml of pre-cooled demineralized water and dried at 45° C. under vacuum (10 mbar) for 48 h. Yellowish vanillin with purity of 99.0% w/w by GC was obtained.

Overall vanillin yields of the processes of Examples 1-4 and 6-13 described herein above were in the range of 40-90%.

1. A process for recovering and purifying vanillin from a microbial fermentation broth, wherein the fermentation broth comprises a vanillin conjugate which is produced during the microbial fermentation by a microbial cell that is capable of producing and secreting the vanillin conjugate, said process comprising the steps of:
(I)(a) separating and removing the microbial cells from the fermentation broth such that a liquid remains that is substantially free of microbial cells followed by converting the vanillin conjugate that is contained in the liquid into vanillin and the corresponding conjugation partner, or
(I)(b) converting the vanillin conjugate that is contained in the fermentation broth into vanillin and the corresponding conjugation partner followed by separating and removing the microbial cells from the fermentation broth such that a liquid remains that is substantially free of microbial cells;
followed by either:
(II)(a) adding a polar organic solvent miscible with water to the liquid resulting from step (I)(a) or (I)(b), wherein the liquid resulting from step (I)(a) is optionally filtered either before or after addition of the polar organic solvent;
(II)(b) treating the vanillin solution produced in step (II)(a) with either:
(i) a cation exchange adsorbent followed by a weak base anion exchange adsorbent, or
(ii) a weak base anion exchange adsorbent followed by a cation exchange adsorbent; and
(II)(c) optionally decolorizing the resulting solution with an adsorbent;
or:
(II′)(a) treating the liquid resulting from step (I)(a) or (I)(b) with a non-ionic adsorbent, wherein the liquid resulting from step (I)(a) is optionally first filtered before treatment with the non-ionic adsorbent, wherein the non-ionic adsorbent used in step (II′)(a) is capable of adsorbing vanillin and wherein the treatment comprises the steps of (i) adsorption of the vanillin under conditions that allow vanillin to bind to the non-ionic adsorbent and (ii) desorption of the bound vanillin into a solution;
(II′)(b) treating the obtained vanillin solution with either:
(i) a cation exchange adsorbent,
(ii) a weak base anion exchange adsorbent, or
(iii) both a cation exchange adsorbent and a weak base anion exchange adsorbent in either order; and
(II′)(c) optionally decolorizing the resulting solution with an adsorbent; and
(III) crystallizing the vanillin from the obtained solution.

2. The process of claim 1, wherein the vanillin conjugate is vanillin glucoside.

3. The process of claim 1, wherein the microbial cell is a fungal cell.

4. The process of claim 3, wherein the fungal cell is a yeast cell.

5. The process of claim 1, wherein the conversion of the vanillin conjugate into vanillin and the corresponding conjugation partner in step (I)(a) or (I)(b) is carried out either by chemical conversion or enzymatic conversion.

6. The process of claim 1, wherein the polar organic solvent used in step (II)(a) is an alcohol or a mixture of different alcohols.

7. The process of claim 1, wherein the cation exchange adsorbent used in step (II)(b) is a strong acid cation exchange adsorbent.

8. The process of claim 1, wherein the weak base anion exchange adsorbent used in step (II)(b) is a weak base anion exchange resin.

9. The process of claim 1, wherein the weak base anion exchange adsorbent used in step (II)(b) is converted to the acetate form before being used.

10. The process of claim 1, wherein the process comprises step (II)(b)(i).

11. The process of claim 1, wherein the adsorbent used in step (II)(c) is carbon.

12. The process of claim 1, wherein the non-ionic adsorbent used in step (II′)(a) is a non-ionic resin.

13. The process of claim 1, wherein desorption of the bound vanillin into a solution in step (II′)(a)(ii) is carried out using an organic solvent, a mixture of different organic solvents, a mixture of water and an organic solvent, or a mixture of water and different organic solvents as eluent.

14. The process of claim 1, wherein the cation exchange adsorbent used in step (II′)(b) is a strong acid cation exchange adsorbent.

15. The process of claim 1, wherein the weak base anion exchange adsorbent used in step (II′)(b) is a weak base anion exchange resin.

16. The process of claim 1, wherein the weak base anion exchange adsorbent used in step (II′)(b) is converted to the acetate form before being used.

17. The process of claim 1, wherein the process comprises step (II′)(b)(iii) and wherein in said step the vanillin solution is treated with a cation exchange adsorbent followed by a weak base anion exchange adsorbent.

18. The process of claim 1, wherein the adsorbent used in step (II′)(c) is carbon.

19. The process of claim 1, wherein the process does not contain a purification step with a strong base anion exchange adsorbent.

20. The process of claim 1, wherein the process occurs at a pH below 7.

21. The process of claim 1, wherein crystallization of vanillin is performed at a pH of about 3.5-5.5.
